(19) United States
(12) Patent Application Publication
(10) Pub. No.: US 2016/0242664 A1
(43) Pub. Date: Aug. 25, 2016

(54) AN APPARATUS AND METHOD FOR EVALUATING MULTICHANNEL ECG SIGNALS

(71) Applicant: KONINKLIJKE PHILIPS N.V., Eindhoven (NL)

(72) Inventors: JIN WANG, SHANGHAI (CN); JINGYI BU, SHANGHAI (CN)

(21) Appl. No.: 15/027,616

(22) PCT Filed: Sep. 25, 2014
(86) PCT No.: PCT/IB2014/064828

§ 371 Date: Apr. 6, 2016

(30) Foreign Application Priority Data
Oct. 9, 2013 (CN) PCT/CN2013/084909
Dec. 2, 2013 (EP) 13195332.5

(51) Int. Cl. A61B 5/04; A61B 5/00; A61B 5/0472; A61B 5/0402; A61B 5/0456

(52) U.S. Cl. CPC A61B 5/04012; A61B 5/0402; A61B 5/04011; A61B 5/0456; A61B 5/0472; A61B 5/7221; A61B 5/7267

(57) ABSTRACT

An evaluating method for ECG signals is provided, which comprises the steps of: a) obtaining a multichannel ECG signal of a subject over a first predetermined time period by means of a multi-lead ECG device; b) extracting a plurality of first parameters from the multichannel ECG signal; c) evaluating the quality of the multichannel ECG signal based on the plurality of first parameters; and d) presenting an indicator for indicating the quality of the ECG signal via a user interface. An ECG signal evaluating apparatus, an ECG signal acquiring apparatus, an ECG signal evaluation system and a computer program are also provided. The ECG signal evaluating method, apparatus, system and computer program of the present invention can improve the accuracy of the evaluation of the quality of the ECG signals and reduce the computational complexity of the evaluation of the quality of the ECG signals.

### FIELD OF THE INVENTION

The present invention relates to acquiring ECG signals, especially to quality evaluation of multichannel ECG signals for mobile health applications.

### BACKGROUND OF THE INVENTION

In addition to the burden of communicable diseases such as malaria, tuberculosis and HIV, developing countries are facing a steady growth in the prevalence of chronic, non-communicable diseases, including heart disease and cancer.

The use of mobile health and/or portable devices to support clinical care provides an opportunity to expand the reach of quality health-care to address both types of disease burdens even in the most remote villages. It is no surprise that mobile health is being touted as the biggest breakthrough in health systems improvement in developing nations. The positive potential for mobile health is huge, but may encounter problems as well.

Expanded and decentralized access to health care results in an increase in demand for expert diagnosis. If the quality of the data needing interpretation is not guaranteed, a loss of efficiency will be foreseen. Consequently, the capacity of the health care system to provide timely expert interpretation will be exceeded, as a result of which not all of the data can be assessed timely.

Recently, some methods have been proposed to assess the quality of ECGs by means of, for example, adopting several rules in time domain, machine learning such as support vector machine (SVM) and artificial neural network, reconstruction matrix, etc. However, they are either of low accuracy or of high computational complexity.

### SUMMARY OF THE INVENTION

Therefore, it would be desirable to increase the efficiency and/or capacity of the mobile health care system making use of ECG signals. It is also desirable to improve the accuracy of the evaluation of the quality of the ECG signals. It would also be desirable to reduce the computational complexity of the evaluation of the quality of the ECG signals.

To better address one or more of these concerns, according to an embodiment of a first aspect of the invention, a method of evaluating a multichannel ECG signal is provided. The method comprises the following steps:

obtaining a multichannel ECG signal of a subject over a first pre-determined time period by means of a multi-lead ECG device;

extracting a plurality of first parameters from the multichannel ECG signal; evaluating the quality of the multichannel ECG signal based on the plurality of first parameters; and

presenting an indicator for indicating the quality of the ECG signal via a user interface.

In this way, a quality indicator is automatically delivered to the user, including the subject person and/or any medical staff such as a nurse, a physician and a doctor, and such a quality indicator can increase the efficiency and/or capacity of the health care system, particularly of the mobile health care system as explained in the following.

Often, the measuring of the ECG signal is performed by a nurse or a physician, who might not be able to evaluate the quality of the measured ECG signal with sufficient accuracy. Sometimes it is noticed at a later point in time, for example by an expert such as a doctor, that the measured ECG signal is not good enough for a meaningful diagnosis, and a re-measurement is required. Such a re-measurement at a later point in time than the first measurement would cost much extra effort and time. Particularly in the mobile health care system, the nurse or the physician measures, by means of a portable device, the ECG signal of the subject at a place far away from where they or the expert are normally located, and therefore, the nurse or the physician might have to travel a long distance to perform the re-measurement when it is decided at a later point in time that the quality of the measured ECG signal is not good enough.

According to the solution proposed herein, the quality of the measured ECG signal is automatically presented via the user interface without requiring any activity by the expert. Accordingly, the patient, the nurse or the physician can be aware of the quality of the measured ECG signal once the measurement is completed. If the quality is not good enough, the re-measurement can be performed immediately after the first measurement.

In one embodiment, the method further comprises the steps of determining whether the quality of the ECG signal satisfies a pre-determined metric, and delivering the ECG signal to a server if the quality of the ECG signal satisfies the pre-determined metric.

In one embodiment, the multichannel ECG signals can be 12-lead ECG signals.

In one embodiment, the plurality of first parameters reflects signal change over time of the multichannel ECG signal and inter-channel correlation of the multichannel ECG signal. By making use of the signal change over time as well as the inter-channel correlation of the multichannel ECG signal, the quality of the ECG signal can be evaluated with enhanced accuracy.

In one embodiment, the step of evaluating the multichannel ECG signal further comprises a step of obtaining at least one second parameter from the plurality of first parameters by means of principal component analysis, and a step of evaluating the quality of the ECG signal, based on the second parameter(s); the number of the at least one second parameter is smaller than the number of the plurality of first parameters.

In one embodiment, the step of evaluating the ECG signal comprises classifying the ECG signal into two or more classes regarding the quality of the ECG signal by means of a classifier.

In one embodiment, the classifier is trained based on a plurality of multi-lead ECG signals and a respective pre-determined classification of each of the plurality of multi-lead ECG signals.

In one embodiment, the step of evaluating comprises mapping the vectors of the first parameters into a space composed of vectors of the second parameter(s) and determining the quality of the ECG signal, based on the distribution of the mapped vectors.

In one embodiment, the plurality of first parameters are calculated by computing mean value and/or deviation of the following basic indices: (1) Inter-channel signal quality index, (2) number of detected beats in each lead of the ECG device, (3) mean correlation value of each PQRST wave in each lead, (4) standard deviation of the correlation value of each PQRST wave in each lead, (5) mean of R-peak to R-peak intervals in each lead, (6) standard deviation of RR intervals in each lead, wherein the ECG signal from each lead of the multi-lead ECG device is divided into segments with a second predetermined period, the basic indices are calculated for each of the segments.

In another aspect, an embodiment of the present invention provides an ECG signal evaluating apparatus, comprising:

a receive unit for receiving a multichannel ECG signal of a subject over a predetermined time period;

a processing device for processing the ECG signal, comprising: an extracting unit for extracting a plurality of first parameters from the ECG signal; and an evaluating unit for evaluating the quality of the ECG signal, based on the plurality of first parameters; and

a user interface for presenting an indicator for indicating the quality of the ECG signal.

In another aspect, an embodiment of the present invention provides an ECG signal acquiring apparatus for acquiring a multichannel ECG signal, wherein the ECG signal acquiring apparatus comprises the above described ECG signal evaluating apparatus.

In another aspect, an embodiment of the present invention provides an ECG signal acquiring system, comprising: one or more ECG signal acquiring apparatuses, at least one of which includes an ECG signal evaluating apparatus as defined above; and a server that is in communication with the ECG signal acquiring apparatuses.

In another aspect, an embodiment of the present invention provides a computer program product comprising a computer program tangibly embodied on a machine readable medium; the computer program, when being executed, is adapted to carry out the method of any one of claims 1 to 8 as defined above.

### DESCRIPTION OF THE DRAWINGS

FIG. 1 is a schematic diagram of ECG signal evaluating apparatus 10 according to an exemplary embodiment of the present invention;

FIG. 2 is a schematic diagram of ECG signal evaluating method 20 according to an exemplary embodiment of the present invention;

FIG. 3 is a schematic diagram of the step of extracting parameters(?) according to an exemplary embodiment of the present invention;

FIG. 4 shows the process of PCA in detail according to an exemplary embodiment of the present invention;

FIG. 5 shows an example of the results of the PCA for a plurality of ECG signals according to an exemplary embodiment of the present invention; and

FIG. 6 shows the process of classifying an ECG signal according to an exemplary embodiment of the present invention.

### DETAILED DESCRIPTION

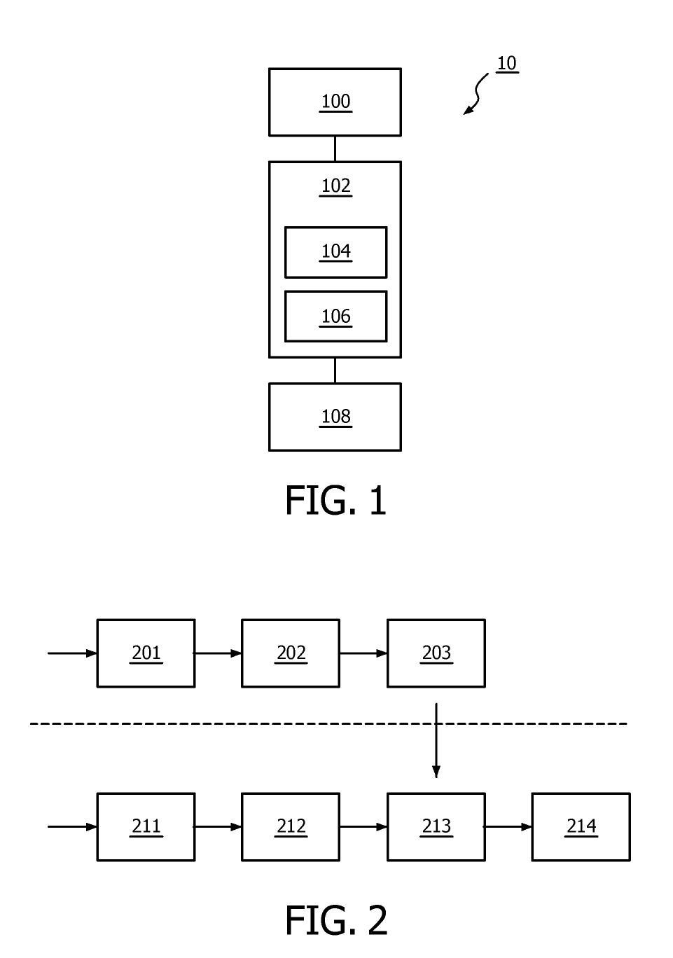

FIG. 1 is a schematic diagram of an ECG signal evaluating apparatus 10 according to an exemplary embodiment of the present invention.

Referring to FIG. 1, the ECG signal evaluating apparatus 10 comprises a receive unit 100 for receiving one or more multichannel ECG signals of a subject, which are acquired during a first predetermined period, such as 60 seconds, 90 seconds or 120 seconds; a processing device 102 for processing the ECG signal; and a user interface 108 for presenting an indicator for indicating the quality of the ECG signal.

In one embodiment, the multichannel ECG signal can be acquired by an ECG acquiring apparatus. The ECG acquiring apparatus can be any ECG device with multiple leads. Subsequently, the acquired signals can be transmitted to the receive unit 100 of the ECG signal evaluation apparatus 10. For example, the ECG device includes 12 leads which are to be attached to a subject at different positions for obtaining signals from the subject. In the case that a physician wants to obtain ECG signals from a subject, he/she attaches the leads of the ECG device to the subject, and the ECG device performs the ECG signal acquiring step, thereby obtaining a respective ECG signal or a set of ECG signals. The ECG signal may be transmitted to the receive unit of the ECG signal evaluating apparatus 10 in various ways using either a wired or wireless connection. The receive unit of the ECG signal evaluating apparatus 10 can be implemented in any suitable manner, such as an input port, a wireless receiver, and so on.

Alternatively, those skilled in the art would understand that, in another embodiment, the ECG signal evaluating apparatus 10 can be part of an ECG acquiring apparatus. In yet another embodiment, the receive unit 100 of the ECG signal evaluating apparatus 10 can be implemented as an ECG acquiring unit with multiple leads for acquiring the ECG signal.

The processing device 102 includes an extracting unit 104 and an evaluating unit 106. The extracting unit 104 is configured to extract a plurality of first parameters (which may be referred to as the first “features” hereinafter) from the ECG signal. For example, the extracting unit may extract 4 to 20 parameters, such as 8, 10, or 12 first parameters, from the ECG signal.

The evaluating unit 106 is configured to evaluate the quality of the ECG signal based on the extracted first parameters. In an example, the evaluating unit 106 is configured to process the first parameters so as to obtain at least one second parameter by applying predetermined transforming coefficients. In another example, the evaluating unit 106 maps vectors, each element of which represents one of the first parameters, into a lower-dimensional space, and evaluates the mapped vectors in the lower-dimensional space. Both the transforming coefficients and the mapping can be either predetermined or derived from a training process. Furthermore, both the transforming coefficients and the mapping can be adapted in the course of evaluating, for example, by means of self-learning.

Then, an indicator is generated and presented via the user interface 108. The indicator indicates the quality of the ECG signal evaluated by the evaluating unit 106. The user interface 108 can present the indicator in visual, audible and/or any other suitable form.

FIG. 2 shows a schematic diagram of an embodiment of the ECG signal evaluating method according to an exemplary embodiment of the present invention. In this embodiment, a training process is carried out to derive information from the training data, such as a set of ECG signals and corresponding quality indicators. The derived information comprises, for example, the transforming coefficients or mapping for transforming the first parameters to the at least one second parameter, and evaluation criteria, a trained classifier or the like for evaluating the quality of the ECG signal based on the at least one second parameter. The training process comprises steps 201, 202 and 203. The training process can be implemented as being performed by the ECG signal evaluating apparatus 10 or, more often, by a separate apparatus, and the data derived from the training process is transmitted to the ECG signal evaluating apparatus 10 in various known ways. Then, in the evaluating process, the multichannel ECG signal is acquired and evaluated based on the data derived from the training process. The evaluating process comprises steps 211, 212, 213 and 214. The evaluating process can be performed by the ECG signal evaluating apparatus 10 as shown in FIG. 1.

At step 201, a set of multichannel ECG signals for training is received together with predetermined evaluation results for each of the multichannel ECG signals. The predetermined evaluation results may be provided by professional physicians, doctors or other medical experts.

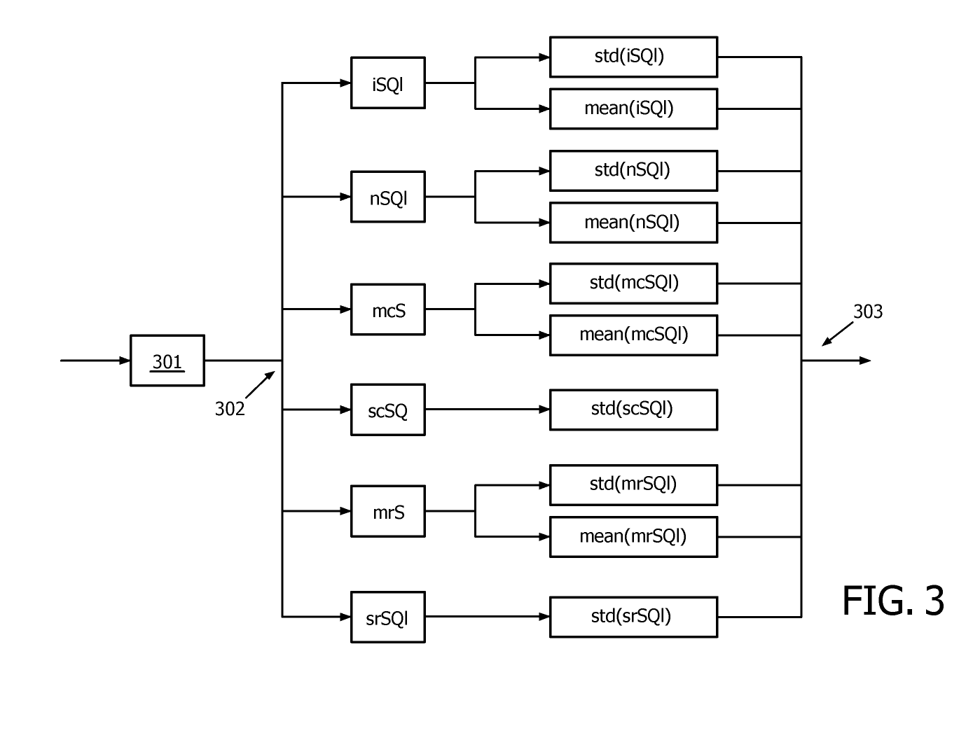

At step 202, each of the multichannel ECG signals in the set is processed, for example by the extracting unit 104, to obtain a plurality of first parameters. The number of the first parameters may be set by a user, e.g., 8, 10, 12, and so on. The first parameters may be parameters/features reflecting the signal change over time of the multichannel ECG signal and inter-channel correlation of the multichannel ECG signal. An exemplary extracting process will be described in detail in the following with reference to FIG. 3.

At step 203, a principal component analysis (PCA) algorithm is applied to the extracted first parameters for each multichannel ECG signal to derive several second parameters from the plurality of first parameters. In one embodiment, the second parameters are principal components of the ECG signals, which represent the characterizations of the ECG signals and will be described in detail in the following. The process of principal component analysis may be based on the first parameters and the evaluation results for each of the ECG signals in the set of ECG signals.

By means of the principal component analysis, a set of transforming coefficients, which will be used to transform the first parameters of a multichannel ECG signal into at least one second parameter, can be obtained. For example, the number of the at least one second parameter can be 2, 3 or 4.

The training process can be performed beforehand, and the results of the training process can be stored in the ECG signal evaluating apparatus 10 or provided to the apparatus 10 in other ways. The training may be performed offline, i.e., on any suitable computer/processor remote from the ECG signal evaluating apparatus 10 of FIG. 1.

In an example, the results of the training process can comprise a set of transforming coefficients for transforming the first parameters into several second parameters, wherein the second parameters may be principal components of the ECG signals. For example, the transforming coefficients may be used to map vectors representing the first parameters into a lower dimensional space composed of principal components of the ECG signals, wherein, the principal components of the ECG signals are also determined by the training process. The results of the training process can further comprise a trained classifier.

At step 211, a multichannel ECG signal of a subject over a first predetermined time period is acquired or received from a multi-lead ECG device.

At step 212, the same plurality of first parameters as in step 202 are extracted from the multichannel ECG signal.

At step 213, the quality of the multichannel ECG signal is evaluated based on the plurality of first parameters. Preferably, at step 213, the plurality of first parameters are transformed into at least one second parameter by a set of predetermined transforming coefficients, wherein the transforming coefficients were predetermined at step 203 and already stored in the ECG evaluating apparatus. Thus, the quality of the multichannel ECG signal is evaluated based on the at least one second parameter.

More preferably, the evaluating unit shown in FIG. 1 includes a classifier, and the evaluating of the quality of the multichannel ECG signal is implemented by the classifier. The classifier is able to classify the multichannel ECG signal, based on the at least one second parameter, into two or more classifications. For example, the classifications can be “acceptable” and “unacceptable” in the case of two classifications, or can be “acceptable”, “unacceptable” and “to be determined” in the case of three classifications. The classifier is also trained during the training process.

At step 214, an indicator to show the quality of the ECG signal, such as the classification of the ECG signal, is presented via a user interface.

Those skilled in the art will appreciate that the training step is not necessarily included in the method of the present invention, which may merely make use of the result of the training process.

As an example, one manner of implementation of the detailed process of the steps of extracting, PCA and classifying will be described hereinbelow. In this example, 10 first parameters are extracted from the multichannel ECG signal.

### Feature Extracting

Actually, the first parameters of the raw ECG signal (for example, 90 seconds, 12-lead) are the derivatives of a number of basic quality indices of each lead. For each lead, a total of 6 basic signal quality indices (SQIs) are calculated. When a set of ECG signals(?) that is measured during a given period, such as, 60 seconds, 90 seconds, and so on, is received/obtained, such an ECG signal is segmented and/or divided into several tens of segments.

Then, as shown in FIG. 3, at 301, beat detection is performed on each of the tens of ECG segments. For each segment, at 302, the positions of QRS are obtained, and then, the 6 basic SQIs will be calculated.

The 6 basic SQIs are given hereinbelow:

1. iSQI: Inter-Channel Signal Quality Index in Each Lead.
When synchronous ECG leads are available, the comparison between different leads can provide more accurate estimates of signal quality. The inter-channel signal quality index iSQI of a lead reflects the percentage/ratio of the number of matched beats detected on a given lead (which beats are matched to the beats detected on another lead) to the number of all the beats that were detected on said given lead and said another lead.
Preferably, the inter-channel signal quality index iSQI of a lead reflects the maximum of said percentage.
In other words, for a given lead, its beats are compared with beats of each of the other leads, and several percentages/ratios are obtained. Then, a maximum percentage/ratio is defined as the Inter-channel signal quality index.
For example, for one lead, such as lead i, the beats detected on this lead are compared with the beats detected on each of the other leads. For example, first, the beats detected on lead i are compared with the beats detected on lead j, wherein j may represent one of the other leads.
And then a ratio of the number of matched beats detected by lead i and matched with lead j (N_{matched}) to all the beats detected (Nₐₗₗ) by lead i and lead j can be calculated, wherein Nₐₗₗ=Nᵢ+Nⱼ−N_{matched} (without double counting the matched beats). This process is repeated for each of the other leads.
Since there may be several other leads, several ratios can be obtained. Then, the maximum of the ratios can be determined. In one embodiment of this invention, such a maximum of the ratios is defined as Inter-channel signal quality index for lead i.
In other words, in this embodiment, the inter-channel signal quality index iSQI for one lead can be calculated as the maximum ratio of the number of matched beats detected by the lead (N_{matched}) and matched with one of the other leads to a sum of the beats (Nₐₗₗ) detected by said lead and said one of the other leads, without double counting the matched beats. That is:

$${iSQI}_{i} = max ( \frac{{N}_{matched ( i , j )}}{{N}_{all ( i , j )}} ) ∀ j , j \neq i$$

wherein i represents the current lead, while j represents each one of the other leads. N_{matched} is the number of beats detected by a lead i and matched with the beats detected by lead j (e.g. within 150 ms) and Nₐₗₗ is the number of all beats detected in the lead i and lead j (without double counting the matched beats).
2. nSQI: The Number of Detected Beats in Each Lead.
3. mcSQI: The Mean Correlation Value of Each PQRST Wave in Each Lead.
After beat detection, every PQRST wave in a certain lead for each 10 s ECG segment is collected and the median template of the PQRST waves is obtained. Then the cross-correlation between each PQRST wave and their median template is calculated. The mean of the cross-correlation values can reflect the quality of a certain lead during its 10 s recording.
   4. scSQI: The Standard Deviation of the Correlation Value of Each PQRST Wave in Each Lead.
The standard deviation of the cross-correlation values between each PQRST wave and their median template can reflect the variation within a certain lead during its 10 s recording, i.e., in a 10 s segment.
   5. mrSQI: The Mean of R-Peak to R-Peak (RR) Intervals in Each Lead.
After beat detection, RR intervals are obtained. The mean of RR intervals in one lead can reflect the quality of this lead. A value of mrSQI which is too small indicates non-detection of beats due to a high noise level or bad recording quality while too large a value of mrSQI means that the QRS complex may be drowned by noise.
   6. srSQI: The Standard Deviation of RR Intervals in Each Lead.
The standard deviation of RR intervals in each lead is calculated for each 10 s segment. A high srSQI implies poor quality of the signal under the assumption that the heart rate of the patient does not vary too much.

The above-described 6 basic signal quality indices are respectively calculated for each segment of the ECG signal. Although in this embodiment, the time period for each segment is selected to be 10 seconds, those skilled in the art will appreciate that the time period for each segment may be, for example, 8 seconds, 12 seconds, and so on.

The extracting unit can obtain the first parameters based on the 6 basic signal quality indices. In other words, the first parameters are derivatives of the 6 basic signal quality indices for the whole ECG signal.

A user can select the number of first parameters to be extracted. For example, in this embodiment, he extracts 10 first parameters. In this embodiment, the extracted 10 first parameters are the mean value (mean) and standard deviation (std) of some of the 6 basic signal quality indices.

As an example, the following 10 parameters/features are chosen:
1. std (mcSQI)
2. std (nSQI)
3. std (iSQI)
4. std (scSQI)
5. std (mrSQI)
6. std (srSQI)
7. mean (nSQI)
8. mean (mcSQI)
9. mean (iSQI)
10. mean (mrSQI).

Therein, “std” represents the standard deviation of the respective basic signal quality indices over all segments, and “mean” represents the mean value of the respective basic signal quality indices over all segments. For example, for each segment, an iSQI value is calculated. Then, the standard deviation of the iSQI values for all segments is calculated and referred to as “std” (iSQI). Similarly, for each segment, mrSQI is calculated, and then, the mean value of the mrSQI over all the segments is calculated and referred to as “mean” (mrSQI).

The 10 first parameters as shown above may be expressed as a 10-dimensional vector. At step 303 in FIG. 3, a 10-dimentional vector “description Vector” composed by the 10 first parameters is formed for the ECG signal with 12 leads and can be utilized to determine whether the ECG signal is good enough for diagnosis (acceptable/unacceptable).

### Principal Component Analysis (PCA)

PCA is used to determine the suitable transforming coefficients for the first parameters, for example, the 10 first parameters mentioned above. In this embodiment, PCA is applied to a training data set of ECG signals. For each of the ECG signals, a 10-dimensional vector representing the 10 first parameters is determined. During the PCA, the 3 largest principal components of the first parameters are determined. Then, the 10-dimensional vector can be mapped into a lower 3-dimentional space composed by the 3 largest principal components, i.e., each one of the three axes of the 3-dimentional space represents one principal component.

The PCA may be performed off-line. Specifically, during the off-line PCA process, a set of ECG signals is provided to a processor for running a parameter extraction and PCA algorithm. Then, said parameter extraction and PCA algorithm are executed on each of the ECG signals in the set of ECG signals.

The PCA method can determine 3 principal components from the 10 first parameters and determines transforming coefficients for each of the 10 features by redundancy removal, which can be used to transform the 10 features into 3 principal components. The PCA algorithm may be a common PCA algorithm.

Below, a description is given in more detail of the process of determining the principal components.

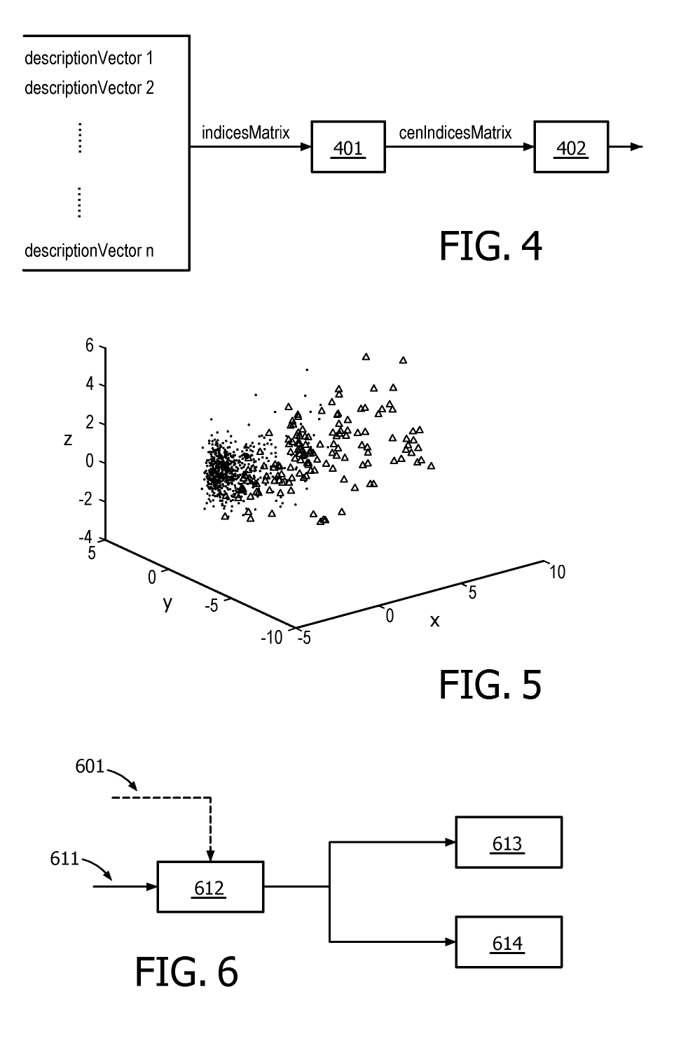

Specifically, while a training data set of ECG signals is provided, the 10-dimensional vector for each of the ECG signals is determined. The number of ECG signals may be “n”, and thus, the number of 10-dimentional vectors “description Vector” is n as well. The n 10-dimensional vectors are collected into a data matrix “indicesMatrix”. With reference to FIG. 4, each row of the data matrix indicates one 10-dimensional vector for one ECG signal. Thus, the matrix is an n*10 matrix, that is, it includes n rows and 10 columns.

Although FIG. 4 merely shows one “descriptionVector” in each row, those skilled in the art should understand that each “descriptionVector” is a 10-dimensional vector, and thus, the “indicesMatrix” actually includes 10 columns, and each column represents one dimension of the “descriptionVector”, which corresponds to one first parameter extracted as mentioned above.

At step 401, the “indicesMatrix” is standardized by subtracting each column by its mean value over the n “descriptionVectors”, before PCA, and a corresponding “cenIndicesMatrix” is obtained.

Then a covariance matrix is calculated as:

covMatrix=cenIndicesMatrix*cenIndicesMatrix^{T}/(n−1)

wherein cenIndicesMatrix^{T} is the transpose of cenIndicesMatrix.

The eigenvectors and eigenvalues of the covariance matrix are calculated. The corresponding eigenvectors of the 3 highest eigenvalues are selected to form the “Feature Vector” , which can be used to apply to the data matrix the cenIndicesMatrix to derive “FinalData” from the first parameters of the processed ECG signal, as:

FinalData=FeatureVector*cenIndicesMatrix^{T}

That is, at step 402, the PCA algorithm is performed on the “cenIndicesMatrix” and the “FinalData” of PCA is obtained. In the PCA, the “FinalData” is a matrix, each row/column of which represents the second parameters of one training ECG signal. In other words, the “FinalData” of PCA represents the second parameters for each ECG signal of the training set. In this embodiment, the second parameters include three parameters. Thus, each data of the “FinalData” includes 3 parameters, i.e., 3 principal components: a first principal component, a second principal component, and a third principal component.

FIG. 5 shows an example of mapping the results of the PCA for a training set including a plurality of ECG signals into a 3-dimensional space composed of the 3 principal components, wherein, “•” represents the ECG signal with the predetermined evaluation result “acceptable” and “Δ” represents the ECG signal with the predetermined evaluation result “unacceptable”. Furthermore, x, y and z axes in FIG. 5 represent the first principal component, the second principal component, and the third principal component, respectively.

For a given unlabeled example “xu”, i.e., for a newly acquired ECG signal, the principal components are obtained based on the transforming coefficients “Feature Vector” as mentioned above. More specifically, 10 first parameters are extracted from the ECG signal, and principal components of the first parameters are determined by mapping the 10 features into a lower 3-dimentional space using the transforming coefficients, wherein the values for 3 principal components of the ECG signal can be used as respective coordinate values in the 3-dimentional space, respectively.

### Classifying

In this embodiment, the K Nearest Neighbor Rule is adopted to classify the ECG signal. The K Nearest Neighbor Rule is a very intuitive method of classifying unlabeled examples (i.e., data representing ECG signals) based on their similarity with examples in a training set.

FIG. 6 shows an example of the process of classifying.

As shown by the portion indicated with a dotted line in FIG. 6, a training step 601 for the classifier 612 is optional. In other words, it may train the classifier 612 based on the same training set as used in PCA, the ECG signals of which have already been classified by professional doctors. Alternatively, the classifier had already been trained, or the classifier had already received training results. For example, in this embodiment, in the classifier there are data representing principal components for each of a large number of training ECG signals together with the classifications for respective training ECG signals. For example, the ECG signals may be classified as “acceptable” or “unacceptable” by the experts or professional doctors. The classifications of the ECG signals may be stored together with respective ECG data (or its principal component computed as given above) in the training ECG data set.

Summarizing, the classifier stores a training data set comprising examples (ECG signals or principal components of the ECG signals) labeled with corresponding classifications.
At 611, a given unlabeled example “xu”, i.e., a newly acquired ECG signal, is provided to the classifier 612, and its position (i.e., coordinate values) in the 3-dimentional space can be determined by using the transforming coefficients. Then, the classifier finds a subset of the labeled examples with k labeled examples that are “closest” to the position of the unlabeled example “xu” in the 3-dimentional space, and assigns “xu” to a classification that appears most frequently within the k-subset.

In this embodiment, the classifier assigns a classification selected from the two classifications, “acceptable” 613 and “unacceptable” 614 to the unlabeled example.

Hereinbelow, the results of Cross-Validation are provided for a set of ECG signals classified by the k-NNR method in the present invention, wherein 500 mobile ECG data streams were evaluated. k was assigned to 7 and the distance was defined to be the Euclidean Distance in the 3-dimensional space. The data in the set of ECG signals are divided into 9 groups and the cross-validation is performed 9 times. Each time, 8 groups of the data are used for training the PCA process and classifying process, and trained transforming coefficients and a trained classifier are obtained, while the last group of data is processed by using the transforming coefficients and is classified by the trained classifier. Such a process is repeated 9 times. Therefore, the validation results for the 9 groups of data are obtained.

The results of Cross-Validation are:

| | 1 | 2 | 3 | 4 | 5 | 6 | 7 | 8 | 9 |
|---|---|---|---|---|---|---|---|---|---|
| Ac- | 0.92 | 0.87 | 0.87 | 0.96 | 0.93 | 0.91 | 0.97 | 0.93 | 0.92 |
| curacy | | | | | | | | | |

The average accuracy is 92.00%.

Obviously, the method and apparatus in the present invention can accurately evaluate the quality of the ECG signal. Based on the automatic feedback on the quality of the ECG signals, ECG signals of unacceptable quality can be easily identified. Accordingly, the ECG signal of unacceptable quality can be removed and/or a re-measurement can be requested.

While the invention has been illustrated and described in detail in the drawings and foregoing description, such illustration and description are to be considered illustrative or exemplary and not restrictive; the invention is not limited to the disclosed embodiments. For example, it is possible to operate the invention in an embodiment in the form of a pair of glasses, a watch or the like.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality.

The present invention can be implemented by means of hardware or software. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems.

1. A non-invasive method of evaluating a multichannel ECG signal, comprising the following steps:
obtaining a multichannel ECG signal of a subject over a first predetermined time period by means of a multi-lead ECG device;
extracting a plurality of first parameters from the multichannel ECG signal;
evaluating the quality of the multichannel ECG signal, based on the plurality of first parameters; and
presenting an indicator for indicating the quality of the ECG signal via a user interface.

2. The method according to claim 1, further comprising the steps of:
determining whether the quality of the ECG signal satisfies a predetermined metric,
delivering the ECG signal to a server if the quality of the ECG signal satisfies the predetermined metric.

3. The method according to claim 1, wherein the plurality of first parameters qualify signal change over time of the multichannel ECG signal and inter-channel correlation of the multichannel ECG signal.

4. The method according to claim 1, wherein the step of evaluating the multichannel ECG signal further comprises a step of obtaining at least one second parameter from the plurality of first parameters, and a step of evaluating the quality of the ECG signal, based on the second parameter(s);
the number of the at least one second parameter is smaller than the number of the plurality of first parameters.

5. The method according to claim 4, wherein the step of evaluating the ECG signal comprises classifying the ECG signal into two or more classes regarding the quality of the ECG signal by means of a classifier.

6. The method according to claim 5, wherein the classifier is trained based on a plurality of multi-lead ECG signals and a respective predetermined classification of each of the plurality of multi-lead ECG signals.

7. The method according to claim 4 wherein the step of evaluating comprises mapping the vectors of the first parameters into a space composed of vectors of the second parameter(s) and determining the quality of the ECG signal, based on the distribution of the mapped vectors.

8. The method according to claim 1, wherein the plurality of first parameters are calculated by computing mean value and/or deviation of the following basic indices: (1) Inter-channel signal quality index, (2) number of detected beats in each lead of the ECG device, (3) mean correlation value of each PQRST wave in each lead, (4) standard deviation of the correlation value of each PQRST wave in each lead, (5) mean of R-peak to R-peak intervals in each lead, (6) standard deviation of RR intervals in each lead, wherein, the ECG signal from each lead of the multilead ECG device is divided into segments with a second predetermined period, the basic indices are calculated for each of the segments.

9. An non-invasive ECG signal evaluating apparatus comprising:
a receive unit for receiving a multichannel ECG signal of a subject over a predetermined time period;
a processing device for processing the ECG signal, comprising:
an extracting unit for extracting a plurality of first parameters from the ECG signal; and
an evaluating unit for evaluating the quality of the ECG signal, based on the plurality of first parameters; and
a user interface for presenting an indicator for indicating the quality of the ECG signal.

10. The ECG signal evaluating apparatus according to claim 9, wherein the evaluating unit is further configured to perform principal component analysis on the plurality of first parameters to obtain at least one second parameter, and the evaluating of the quality of the ECG signal is based on the principal components, and
wherein the number of the at least one second parameter is smaller than the number of the plurality of first parameters.

11. The ECG signal evaluating apparatus according to claim 9, wherein the evaluating unit includes a classifier for classifying the ECG signal regarding the quality of the ECG signal.

12. The ECG signal evaluating apparatus according to claim 11, wherein the vectors of the first parameters are mapped into a space composed of vectors of the second parameter(s), and the classifier is configured to determine the quality of the ECG signal based on the distribution of the mapped vectors.

13. An ECG signal-acquiring apparatus for acquiring a multichannel ECG signal, wherein the ECG signal-acquiring apparatus comprises the ECG signal-evaluating apparatus according to claim 9.

14. An ECG signal-evaluation system, comprising:
one or more ECG signal-acquiring apparatus, at least one of which includes an ECG signal-evaluating apparatus as defined in claim 9; and
a server that is in communication with the ECG signal-acquiring apparatus.

15. A computer program product comprising a computer program tangibly embodied on a machine-readable medium, the computer program, when being executed, is adapted to carry out the method of claim 1.
